(19) United States
(12) Patent Application Publication
(10) Pub. No.: US 2019/0216362 A1
(43) Pub. Date: Jul. 18, 2019

(54) SENSING DEVICE FOR POSITIONING AFFECTED PART AND METHOD THEREFOR

(71) Applicants: GIL MEDICAL CENTER, Incheon (KR); GACHON UNIVERSITY OF INDUSTRY-ACADEMIC COOPERATION FOUNDATION, Gyeonggi-do (KR)

(72) Inventor: Jun Won Chung, Seoul (KR)

(21) Appl. No.: 16/320,968

(22) PCT Filed: Jun. 28, 2017
(86) PCT No.: PCT/KR2017/006811

§ 371 Date: Jan. 25, 2019

(30) Foreign Application Priority Data
Jul. 25, 2016 (KR) 10-2016-0094384

(51) Int. Cl. A61B 5/06; A61B 90/00; A61B 5/00; A61B 1/00

(52) U.S. Cl. CPC A61B 5/064; A61B 90/39; A61B 5/7405; A61B 5/742; A61B 2090/3975; A61B 2090/3912; A61B 2090/3987; A61B 2090/3958; A61B 1/00087

(57) ABSTRACT

Disclosed are a detection device and a detection method for recognition of a location of an affected part. The detection device includes: a clip (100) attached to an affected part through an endoscopic tool and including an electronic marker (110); and a probe (200) including a measurement unit (210) configured to recognize the electronic marker (110) and which approaches the affected part from an outside of the affected part.

### TECHNICAL FIELD

The present invention relates to a detection device and a detection method for recognition of a location of an affected part, and more particularly to a detection device and a detection method for accurately recognizing a location, at which an affected part has been removed during an endoscopic operation, during a laparoscopic or surgical operation which is performed after the endoscopic operation, by attaching a clip to the location at which the affected part has been removed during the endoscopic operation.

### BACKGROUND ART

A polypus may be immediately removed during an inspection, by inserting an endoscope into a human body, in a gastroenterological clinic. However, when a surgical operation, such as a laparoscopic surgery, is further necessary due to the size or hardness of a polypus, it is necessary to indicate a location of the polypus, which has been removed by another surgical operator, that is, a location of the affected part during an endoscopic operation. However, a tool approaches an affected part from an inside of an organ, such as a stomach or a large intestine in the endoscopic operation, whereas a tool approaches an affected part from an outside of an organ in the surgical operation so that it is not easy to inform the affected part.

In order to solve the problems, there has been a method of indicating a location through an endoscopic injection therapy by using India ink, Indocyanine green, or Indigocarmine. According to the method, there is a risk of obstructing clear view of lesion or generating a peritonitis during a surgical operation.

Further, there has been a method of indicating a location through an injection therapy by using the blood of a patient instead of using the ink. The method may prevent generation of a peritonitis because it uses the blood of a patient, but there is a problem of obstructing a view of a lesion during a surgical operation.

The related conventional technologies are as follows.

Korean Patent Application Publication No. 2015-0070089 discloses a technology using a magnetic marker for setting a surgical location, but there is a limit in applying the technology to an affected part that is located deep in a human body because the technology is applied not to an endoscopic surgery but to a biopsy.

Korean Patent Application Publication No. 2011-0071976 discloses an endoscopic clip for indicating a location by using a radioactive material to set a surgical location, but there is a danger of a radioactive exposure when a radioactive material is used.

Korean Patent Application Publication No. 2013-0113007 discloses an endoscopic clip for indicating a location, which includes a light emitting element to set a surgical location, there is a danger factor due to a malfunction of an electronic component because the electronic component is essential for light emission.

### DISCLOSURE

### Technical Problem

The present invention provides a detection device and a detection method for detecting a location of an affected part, by which a location of a patient may be accurately recognized from an outside of the affected part during a laparoscopic or surgical operation while not do harmful to a human body.

### Technical Solution

In accordance with an aspect of the present invention, the invention provides a detection device including: a clip 100 attached to an affected part through an endoscopic tool and including an electronic marker 110; and a probe 200 including a measurement unit 210 configured to recognize the electronic marker 110 and which approaches the affected part from an outside of the affected part.

It is preferable that the clip 100 is formed of a flexible material and has a Y shape.

It is preferable that the electronic markers 110 is at least one electronic marker 110 located on an outer peripheral surface of a distal end of the clip 100.

The measurement unit 210 may be configured to recognize the electronic marker 110 to identify a location of the affected part from the outside.

It is preferable that the electronic marker 110 includes a magnetic material 110a or a metallic material 110b or includes an RFID tag 110c.

It is preferable that if the electronic marker 110 includes the magnetic material 110a, the measurement unit 210 includes a metallic material 210a.

It is preferable that the detection device further includes: an output unit 300 configured to output information acquired by the measurement unit 210 through a preset method.

It is preferable that if the electronic marker 110 includes the metallic material 110b, the measurement unit 210 includes a metal detection unit 210b.

It is preferable that if the electronic marker 110 includes the RFID tag 110c, the measurement unit 210 includes an RFID reader 210c.

The output unit 300 may include a speaker 300b or a display unit 300c, and the speaker 300b or the display unit 300c may be operated if a value detected by the metal detection unit 210b is a preset value or more.

The output unit 300 may include a display unit 300c, and if the RFID reader 210c recognizes the RFID tag 110c, the display unit 300c may be operated.

In accordance with another embodiment of the present invention, the invention provides a detection method using a detection device including: (a) attaching a clip 100 mounted on an endoscopic tool and including an electronic marker 110 to a specific location of an affected part and retrieving the endoscopic tool; and (b) allowing a probe 200 including a measurement unit 210 capable of recognizing the electronic marker 110 to approach the specific location from an outside of the affected part to recognize the electronic marker 110.

In (a), the electronic marker 110 may include a magnetic material 110a, and in (b), the measurement unit 210 includes a metallic material 210a, and the location of the affected part may be detected through an interaction between the magnetic material 110a and the metallic material 210a.

It is preferable that the detection method further includes: (c) outputting information acquired by the measurement unit 210 through a preset method, by an output unit 300.

In (a), the electronic marker 110 may include a metallic material 110b, and in (b), the measurement unit 210 may include a metal detection unit 210b, and the location of the affected part may be detected through a metal detection function of the metal detection unit 210b.

In (a), the electronic marker 110 may include an RFID tag 110c, and in (b), the measurement unit 210 includes an RFID reader 210c, and the location of the affected part may be detected through recognition of the RFID tag 110c of the RFID reader 210c.

### Advantageous Effects

According to the detection device of the present invention, an accurate location of a portion, which is to be cut away, may be easily and promptly recognized during a laparoscopic or surgical operation by adding an electronic marker to an existing bio-clip to reinforce a function as a location indicator in addition to a basic function of a clip, and the accuracy of a surgery may be increased and a side-effect may be minimized by allowing the location of the affected part to be found through audible and visual elements.

Further, according to the detection device according to the present invention, an operation and a surgery may be safely performed because the detection device does not use a radioactive material that is harmful to a human body.

Further, because a clip attachment operation may be performed by directly using an existing endoscopic device, the technology is cost-efficient as it may be used without having to introduce a separate system during an operation.

### DESCRIPTION OF THE INVENTION

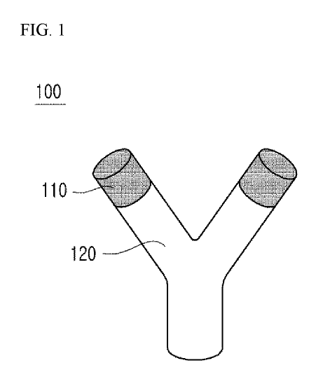

FIG. 1 is a perspective view of a clip according to the present invention;

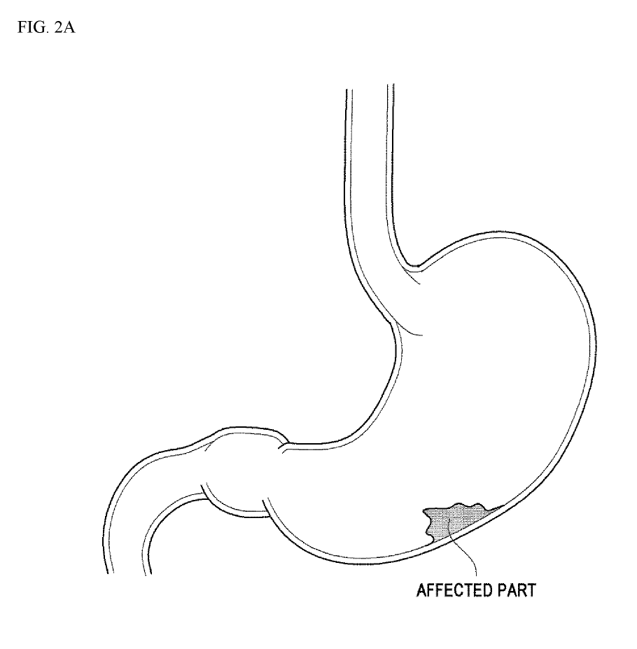

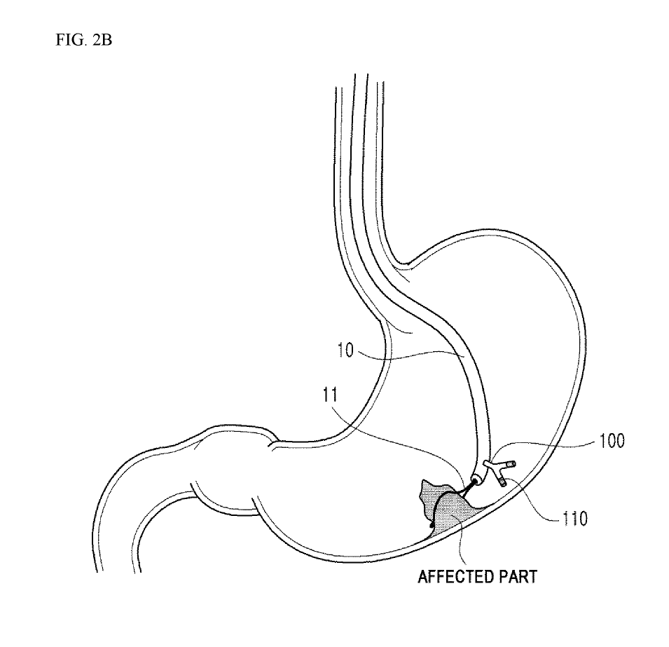

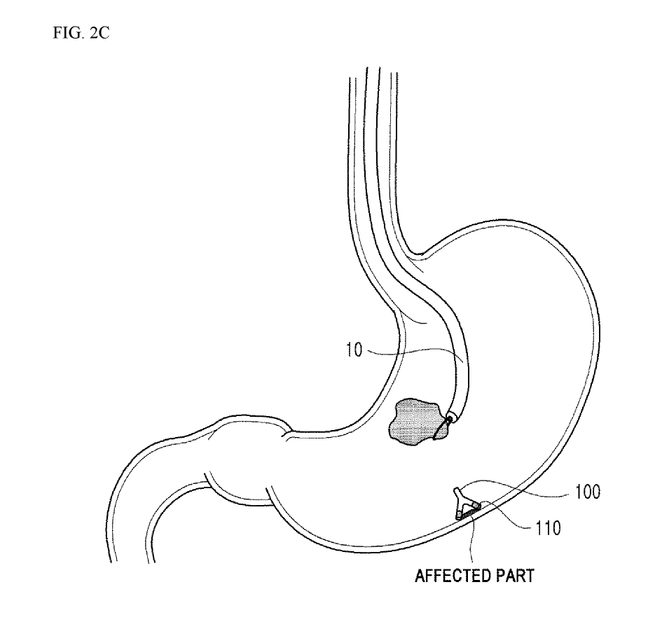

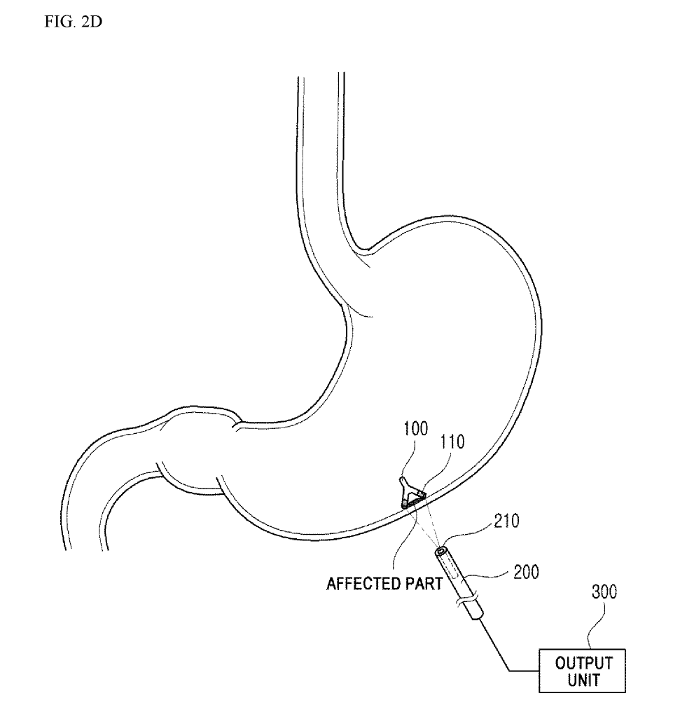

FIG. 2A illustrates a state in which an affected part is located at a specific location of a human body;

FIG. 2B illustrates a state in which the affected part is cut away through a snare of an endoscopic tool;

FIG. 2C illustrates a state in which the clip of FIG. 1 is attached to the cutaway location of the affected part and the endoscopic tool is retrieved, after the affected part is cut away;

FIG. 2D illustrates a state in which an electronic marker and a measurement unit interact with each other to trace a location of the affected part according to an embodiment of the present invention; and

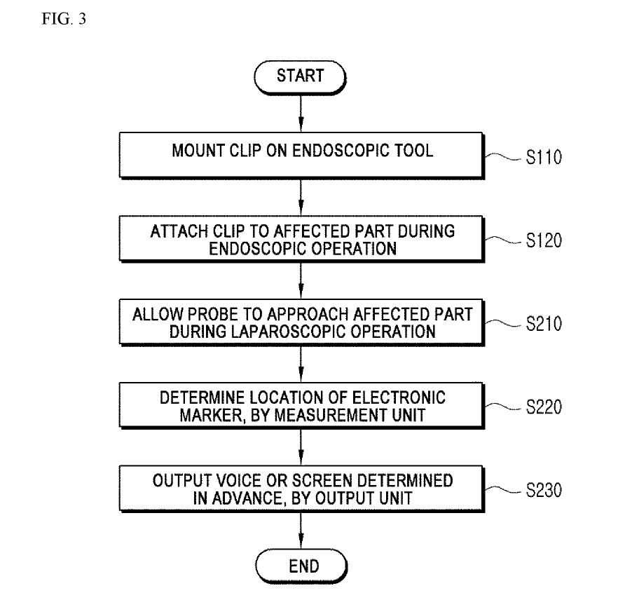

FIG. 3 illustrates a flowchart for tracing a location of the affected part according to an embodiment of the present invention.

### BEST MODE

1. Description of Terms

An inside and an outside of an affected part described in the specification of the present invention will be described. With respect to an affected part, ‘an inside of the affected part’ corresponds to a step, through which an endoscopic tool approaches the affected part after an endoscope is inserted into a human body during an endoscopic operation, and ‘an outside of the affected part’ corresponds to a step, through which a laparoscopic tool or a surgical tool approaches to the affected part during a laparoscopic or surgical operation.

2. Overall Description of Detection Device

Hereinafter, a detection device according to an embodiment of the present invention will be described.

Referring to FIGS. 1 and 2D, the detection device according to the embodiment of the present invention includes a clip 100 attached to an inside of an affected part and a probe 200 for recognition of the clip 100 from an outside of the affected part.

The clip 100 is attached to a location at which the affected part has been removed during an endoscopic operation, and functions as a surgery location indicator that informs a location of the affected part during a laparoscopic or surgical operation. It is preferable that the clip 100 be formed of a flexible material and have a Y shape. As the clip 100 is formed of a flexible material and has a Y shape, the width of the clip 100 may be adjusted and the clip 100 may be easily attached to the affected part.

The clip 100 includes an electronic marker 110 that may interact with a measurement unit 210, which will be described below. At least one electronic marker 110 is located on an outer peripheral surface of a distal end of the clip 100. As the electronic marker 110 is located at the distal end of the clip 100, a distance between the measurement unit 210 and the electronic marker 110 becomes shorter and an interaction between the measurement unit 210 and the electronic marker 110 becomes easy, and as a result, it becomes easy to identity a location of the affected part from an outside of the patient.

The electronic marker 110 includes a magnetic material 110a or a metallic material 110b or includes an RFID tag 110c. Accordingly, the location of the affected part may be identified from the outside of the affected part through an interaction between the electronic marker 110 and a metallic material 210a, a metallic detection part 210b, and an RFID reader 210c included in the measurement unit 210, which will be described below.

The detection device according to the embodiment of the present invention further includes an output unit 300 that outputs information. The output unit 300 outputs the information acquired by the measurement unit 210 through a preset method. The output unit 300 includes a speaker 300b that outputs information through sounds or a display unit 300c that outputs information through a screen. Accordingly, during a laparoscopic or surgical operation, it may be audibly or visually identified whether a surgical tool is accurately located at the location of the affected part.

3. Description of Detection Device According to First Embodiment

Hereinafter, a detection device according to a first embodiment of the present invention will be described in detail.

In the detection device according to the first embodiment, the electronic marker 110 includes a magnetic material 110a. Then, because the measurement unit 210 includes a metallic material 210, the magnetic material 110a and the metallic material 210a get closer to each other through an interaction (for example, an attractive force between the S pole and the N pole of the magnet) of the magnetic material 110a and the metallic material 210a even though a force is not applied. Through, the location of the affected part may be accurately recognized during the laparoscopic or surgical operation.

4. Description of Detection Device According to Second Embodiment

Hereinafter, a detection device according to a second embodiment of the present invention will be described in detail.

In the detection device according to the first embodiment, the electronic marker 110 includes a metallic material 110b.

Then, because the measurement unit 210 includes a kind of a small-sized metal detection unit 210b having a function of detecting a metal, a location of the electronic marker 110 including the metallic material 110b may be recognized.

The metal detection unit 210b detects the metallic material 110b through a method of allowing currents to flow directly through the metal detection unit 210b for a short period of time and measuring a residual magnetic field of the metal detection unit 210b after the currents are interrupted. Because the magnetic field stays in the metal detection unit 210b for a longer period of time as the distance between the metal detection unit 210b and the metallic material 110b becomes shorter, a value detected by the metal detection unit 210b becomes larger as the distance between the metal detection unit 210b and the metallic material 110b becomes shorter.

Because the detection device according to the second embodiment includes an output unit 300, the speaker 300b or the display unit 300c may be operated when the value detected by the metal detection unit 210b is a preset value or more so that it may be audibly or visually identified whether the surgical tool is accurately located at the location of the affected part during a laparoscopic or surgical operation.

5. Description of Detection Device According to Third Embodiment

Hereinafter, a detection device according to a third embodiment of the present invention will be described in detail.

In the detection device according to the third embodiment, the electronic marker 110 includes an RFID tag 110c.

Then, because the measurement unit 210 includes an RFID reader 210c that may recognize the RFID tag 110c, the location of the electronic marker 110 including the RFID tag 110c may be recognized.

Because the detection device according to the third embodiment includes the output unit 300, the display unit 300c may be operated so that it may be visually identified whether the surgical tool is accurately located at the location of the affected part during the laparoscopic or surgical operation when the RFID reader 210c recognizes the RFID tag 110c.

6. Description of Detection Method Using Detection Device

Hereinafter, a detection method using the detection device according to an embodiment of the present invention will be described in detail with reference to FIG. 2A to 2D.

FIG. 2A illustrates a state in which an affected part is located at a specific location of a human body.

FIG. 2B illustrates a state in which the affected part has been cut away by using an endoscopic device such as a snare 11, by an endoscopic tool 10. Because the endoscopic tool 10 includes a clip 100 that may be attached for display of the surgical location (S110), the clip 100 is attached at the location at which the affected part has been cut away (S120). The endoscopic tool 10 is retrieved after the clip 100 is attached to the location of the affected part, and as a result, the clip 100 is attached to the inside of the affected part (see FIG. 2C).

Referring to FIG. 2D, when a surgical operator other than the endoscopic operator requires a laparoscopic or surgical operation, a laparoscopic tool or a surgical tool is inserted into the human body. Then, the surgical tool includes a probe 200 to recognize the location of the affected part. The probe 200 traces the location of the patient while approaching the affected part from the outside of the affected part (S210).

Because the measurement unit 210 included in the probe 200 may interact with the electronic marker 110 of the clip 100 according to the kind of the electronic maker 110, the location of the electronic marker 110 may be determined (S220), and through this, the location of the affected part may be accurately recognized.

Because the electronic marker 110 includes the magnetic material 110a and the measurement unit 210 includes the metallic material 210a in the detection device according to the first embodiment of the present invention, the magnetic material 110a and the metallic material 210a get closer to each other through the interaction (for example, an attractive force between the S pole and the N pole of the magnet) of the magnetic material 110a and the metallic material 210a even though a force is not applied. Because the measurement unit 210 determines the location of the electronic marker 110 through this (S220), the location of the affected part may be accurately recognized during a laparoscopic or surgical operation.

The detection method using the detection device according to the embodiment of the present invention further includes an operation of outputting information acquired by the measurement unit 210 through a preset method, by the output unit 300.

When the measurement unit 210 determines the location of the electronic marker 110, the output unit 300 outputs the information acquired by the measurement unit 210 through a preset method. The output unit 300 includes a speaker 300b that outputs information through sounds or a display unit 300c that outputs information through a screen. Accordingly, during a laparoscopic or surgical operation, it may be audibly or visually identified whether a surgical tool is accurately located at the location of the affected part (S230).

In the detection device according to the second embodiment of the present invention, because the electronic marker 110 includes the metallic material 110b and the measurement unit 210 includes a kind of a small-sized metal detection unit 210b having a function of detecting a metal, the location of the electronic marker 110 including the metallic material 110b may be determined (S220). The speaker 300b or the display unit 300c may be operated when the value detected by the metal detection unit 210b is a preset value or more so that it may be audibly or visually identified whether the surgical tool is accurately located at the location of the affected part during the laparoscopic or surgical operation (S230).

In the detection device according to the third embodiment of the present invention, because the electronic marker 110 includes the RFID tag 110c and the measurement unit 210 includes the RFID reader 210c that may recognize the RFID tag 110c, the location of the electronic marker 110 including the RFID tag 110c may be determined. When the RFID reader 210c recognizes the RFID tag 110c, the display unit 300c may be operated so that it may be visually identified whether the surgical tool is accurately located at the location of the affected part during a laparoscopic or surgical operation (S230).

Although the embodiment of the present invention illustrated in the drawings has been described so that those skilled in the art can easily reproduce and implement the present invention, it is merely exemplary and it will be understood by those skilled in the art that various modifications and equivalent embodiments can be made. Therefore, the scope of the present invention should be determined according to the claims.

### DESCRIPTION OF REFERENCE NUMERALS

10: Endoscopic tool

11: Snare

100: Clip

110: Electronic marker

110a: Magnetic material

110b: Metallic material

110c: RFID tag

200: Probe

210: Measurement unit

210a: Metallic material

210b: Metal detection unit

210c: RFID reader

1. A detection device comprising:
a clip (100) including an electronic marker (110) configured to be attached to an affected part through an endoscopic tool and; and
a probe (200) including a measurement unit (210) configured to recognize the electronic marker (110) and approach the affected part from an outside of the affected part.

2. The detection device of claim 1, wherein the clip (100) is formed of a flexible material and has a Y shape.

3. The detection device of claim 2, wherein the electronic markers (110) is at least one electronic marker (110) located on an outer peripheral surface of a distal end of the clip (100).

4. The detection device of claim 1, wherein the measurement unit (210) is configured to recognize the electronic marker (110) to identify a location of the affected part from the outside.

5. The detection device of claim 1, wherein the electronic marker (110) includes a magnetic material (110a) or a metallic material (110b), or includes an RFID tag (110c).

6. The detection device of claim 5, wherein if the electronic marker (110) includes the magnetic material (110a), the measurement unit (210) includes a metallic material (210a).

7. The detection device of claim 5, further comprising:
an output unit (300) configured to output information acquired by the measurement unit (210) through a predetermined method.

8. The detection device of claim 7, wherein if the electronic marker (110) includes the metallic material (110b), the measurement unit (210) includes a metal detection unit (210b).

9. The detection device of claim 7, wherein if the electronic marker (110) includes the RFID tag (110c), the measurement unit (210) includes an RFID reader (210c).

10. The detection device of claim 8, wherein the output unit (300) includes a speaker (300b) or a display unit (300c), and
wherein the speaker (300b) or the display unit (300c) is operated when a value detected by the metal detection unit (210b) is a preset value or more.

11. The detection device of claim 9, wherein the output unit (300) includes a display unit (300c), and
wherein if the RFID reader (210c) recognizes the RFID tag (110c), the display unit (300c) is operated.

12. A detection method using a detection device comprising:
(a) attaching a clip (100) including an electronic marker and mounted on an endoscopic tool to a specific location of an affected part, and retrieving the endoscopic tool; and
(b) allowing a probe (200) including a measurement unit (210) capable of recognizing the electronic marker (110) to approach the specific location from an outside of the affected part to recognize the electronic marker (110).

13. The detection method of claim 12, wherein in (a), the electronic marker (110) includes a magnetic material (110a), and in (b), the measurement unit (210) includes a metallic material (210a), and
wherein the location of the affected part is detected through an interaction between the magnetic material (110a) and the metallic material (210a).

14. The detection method of claim 12, further comprising:
(c) outputting information acquired by the measurement unit (210) through a predetermined method, by an output unit (300).

15. The detection method of claim 14, wherein in (a), the electronic marker (110) includes a metallic material (110b), and in (b), the measurement unit (210) includes a metal detection unit (210b), and
wherein the location of the affected part is detected through a metal detection function of the metal detection unit (210b).

16. The detection method of claim 14, wherein in (a), the electronic marker (110) includes an RFID tag (110c), and in (b), the measurement unit (210) includes an RFID reader (210c), and
wherein the location of the affected part is detected through recognition of the RFID tag (110c) by the RFID reader (210c).
